(19) United States
(12) Patent Application Publication
(10) Pub. No.: US 2002/0106788 A1
(43) Pub. Date: Aug. 8, 2002

(54) Permeable reactor plate and method

(76) Inventor: James Norman Cawse, Pittsfield, MA (US)

Correspondence Address: General Electric Company, CRD Patent Docket Rm 4A59, P.O. Box 8, Bldg. K-1 - Salamone, Schenectady, NY, 12301, US

(21) Appl. No.: 09/729,118

(22) Filed: Dec. 4, 2000

(51) Int. Cl. G01N 33/53; C12M 1/22

(52) U.S. Cl. 435/305.2; 422/99

(57) ABSTRACT

A reactor plate comprises a substrate with an array of reaction cells and a permeable film covering at least one of the cells to selectively permit transport of a reactant gas into the one cell while preventing transport of a reaction product out of the cell. A method comprises providing a reactor plate comprising a substrate with an array of reaction cells, at one least one cell of the array comprising a cavity and a permeable film cover and conducting a combinatorial high throughput screening (CHTS) method with the reactor plate.

### BACKGROUND OF THE INVENTION

The present invention relates to a reactor plate and method for running multiple parallel screening reactions with multiphase reactant systems.

In experimental reaction systems, each potential combination of reactant, catalyst and condition must be evaluated in a manner that provides correlation to performance in a production scale reactor. Combinatorial organic synthesis (COS) is a high throughput screening (HTS) methodology that was developed for pharmaceuticals. COS uses systematic and repetitive synthesis to produce diverse molecular entities formed from sets of chemical “building blocks.” As with traditional research, COS relies on experimental synthesis methodology. However instead of synthesizing a single compound, COS exploits automation and miniaturization to produce large libraries of compounds through successive stages, each of which produces a chemical modification of an existing molecule of a preceding stage. A library is a physical, trackable collection of samples resulting from a definable set of processes or reaction steps. The libraries comprise compounds that can be screened for various activities.

The technique used to prepare such libraries involves a stepwise or sequential coupling of building blocks to form the compounds of interest. For example, Pirrung et al., U.S. Pat. 5,143,854 discloses a technique for generating arrays of peptides and other molecules using light-directed, spatially-addressable synthesis techniques. Pirrung et al. synthesizes polypeptide arrays on a substrate by attaching photoremovable groups to the surface of the substrate, exposing selected regions of the substrate to light to activate those regions, attaching an amino acid monomer with a photoremovable group to the activated region and repeating the steps of activation and attachment until polypeptides of desired lengths and sequences are synthesized.

Combinatorial high throughput screening (CHTS) is an HTS methodology that incorporates characteristics of COS. The definition of the experimental space permits a CHTS investigation of highly complex systems. The method selects a best case set of factors of a chemical reaction. The method comprises defining a chemical experimental space by (i) identifying relationships between factors of a candidate chemical reaction space; and (ii) determining a chemical experimental space comprising a table of test cases for each of the factors based on the identified relationships between the factors with the identified relationships based on researcher specified n-tuple combinations between identities of the relationships. A CHTS method is effected on the chemical experimental space to select a best case set of factors.

The methodology of COS is difficult to apply in certain reaction systems. For example up to now, COS has not been applied to systems that may produce vaporous products that may escape from respective cells of an array and contaminate the contents of adjacent or near-by cells. There is a need for improved reaction plate and method to permit rapid and effective investigation of vaporous product reaction systems.

### BRIEF SUMMARY OF THE INVENTION

The invention provides a reactor plate and method to investigate these types of systems. According to the invention, a reactor plate comprises a substrate with an array of reaction cells and a permeable film covering at least one of the cells to selectively permit transport of a reactant gas into the one cell while preventing transport of a reaction product out of the cell.

A method comprises providing a reactor plate comprising a substrate with an array of reaction cells, at one least one cell of the array comprising a cavity and a permeable film cover and conducting a combinatorial high throughput screening (CHTS) method with the reactor plate.

### BRIEF DESCRIPTION OF THE DRAWINGS

FIG. 1 is a schematic representation of a top view of a reactor plate according to the invention;

FIG. 2 is a schematic cut-away front view through line A-A of the reactor plate of FIG. 1;

FIGS. 3 to 5 are schematic cut-away representations of various cell configurations;

FIG. 6 is a graph of permeability versus film thickness;

FIG. 7 is a graph of permeability versus temperature; and

FIG. 8 is a 3-D column graph showing interations of transition metal cocatalysts with lanthanide metal cocatalysts.

### DETAILED DESCRIPTION OF THE INVENTION

In an embodiment, the invention is directed to a reactor plate and method for CHTS. The method and system of the present invention can be useful for parallel high-throughput screening of chemical reactants, catalysts, and related process conditions.

Typically, a CHTS method is characterized by parallel reactions at a micro scale. In one aspect, CHTS can be described as a method comprising (A) an iteration of steps of (i) selecting a set of reactants; (ii) reacting the set and (iii) evaluating a set of products of the reacting step and (B) repeating the iteration of steps (i), (ii) and (iii) wherein a successive set of reactants selected for a step (i) is chosen as a result of an evaluating step (iii) of a preceding iteration.

In another typical CHTS method, a multiplicity of tagged reactants is subjected to an iteration of steps of (A) (i) simultaneously reacting the reactants, (ii) identifying a multiplicity of tagged products of the reaction and (B) evaluating the identified products after completion of a single or repeated iteration (A).

A typical CHTS can utilize advanced automated, robotic, computerized and controlled loading, reacting and evaluating procedures.

These and other features will become apparent from the drawings and following detailed discussion, which by way of example without limitation describe preferred embodiments of the present invention.

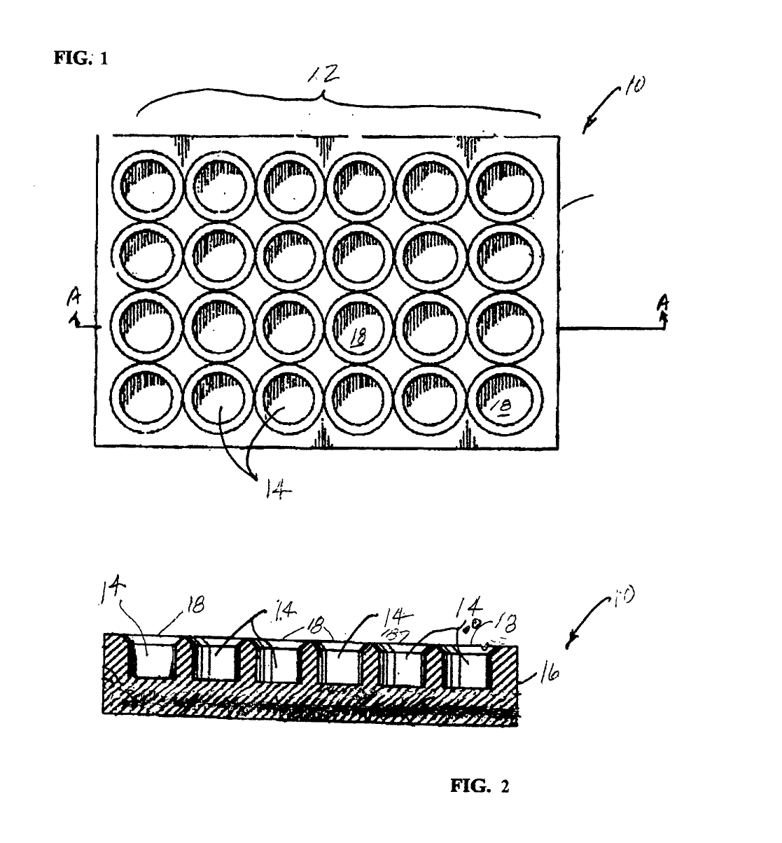

FIG. 1 shows a top view of a preferred reactor plate and FIG. 2 shows a cut-away front view through line A-A of the plate of FIG. 1. FIG. 1 and FIG. 2 show reactor plate 10 that includes an array 12 of reaction cells 14 embedded into a supporting substrate 16 of the plate 10. Each cell 14 is shown covered with a permeable film 18. Each cell 14 can be covered with the same film 18 or each cell can be covered with a different film to provide different reaction characteristics to different cells 14. Further, in another embodiment, selected cells 14 can be covered with film while other cells 14 are left uncovered to provide still different reaction characteristics.

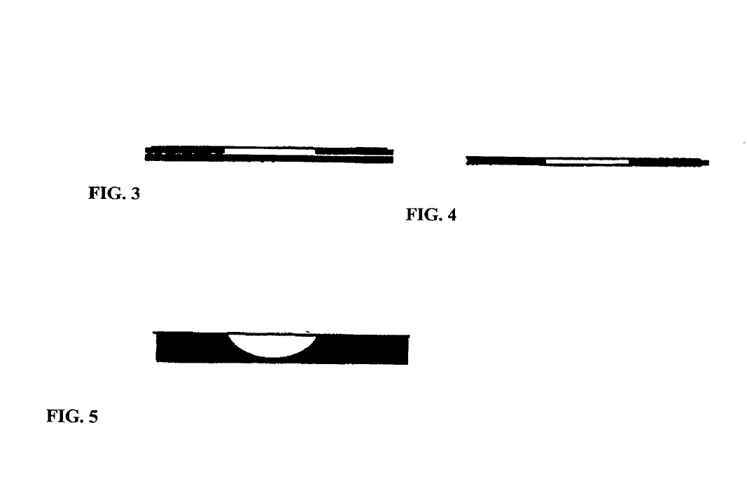

FIGS. 3, 4 and 5 illustrate embodiments of the cell of the invention. FIG. 3 shows a shallow cell with permeable film cover. For example, the cell can have a volume of about 20 mm³, a film area of 20 mm², a 1 mil film and a 1 mm deep cavity. FIG. 4 shows a cell with two opposing walls comprising permeable film. For example, the cell can have a volume of about 20 mm³, a film area of 40 mm², a 1 mil film and a 1 mm deep cavity. FIG. 5 shows a concave bottomed cell with permeable film cover. For example, the cell can have a volume of about 40-50 mm³, a film area of 2-3 mm², a 1 mil film and a 5 mm deep cavity. The respective cells and films are selected by considering permeability of the film and robustness and rate of the reaction. For example, the cells can be designed so that rate of diffusion of gas through the membrane is greater than the rate of gas uptake of the reaction. In this instance, the system would be “reaction-limited” rather than “diffusion-limited.”

The film 18 can be any permeable film that will selectively admit transport of a reactant but will prohibit transport of a reaction product in a CHTS process. For example, the film can be a polycarbonate, perfluoroethylene, polyamide, polyester, polypropylene, polyethylene or a monofilm, coextrusion, composite or laminate.

Polycarbonate, PET and polypropylene are preferred films. Relative humidity may affect permeability of many films. However, permeability of polycarbonate, PET and polypropylene is substantially unaffected by changes in humidity. Hence, these films are particularly advantageous to conduct reactions in humid conditions or to conduct moisture sensitive reactions such as a carbonylation reaction.

In certain applications, the film can be characterized by a diffusion coefficient of about 5×10⁻¹⁰ to about 5×10⁻⁷, desirably about 1×10⁻⁹ to about 1×10⁻⁷ and preferably about 2×10⁻⁸ to about 2×10⁻⁶ in units of cc(STP)-mm/cm²-sec-cmHg.

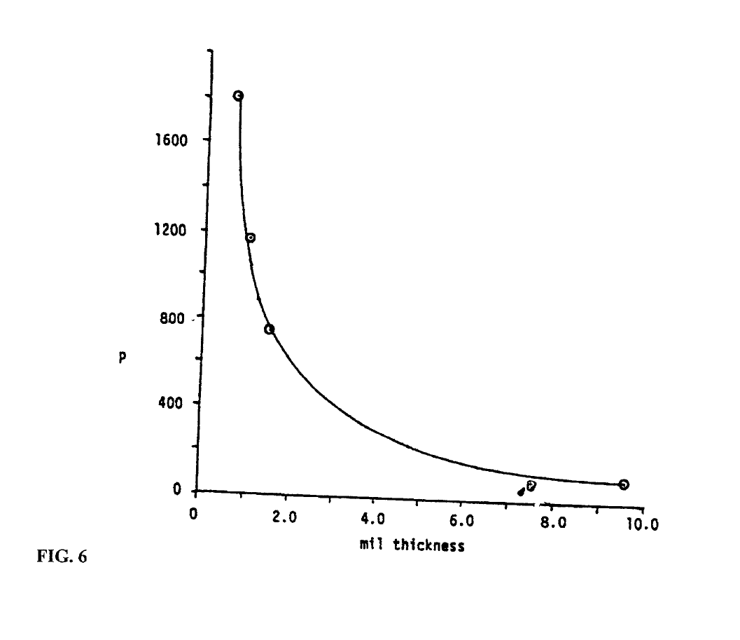

The permeability of a film will vary with thickness. In this invention, the film can be of any thickness that will admit transport of a reactant, usually a gas or vapor, but that will prohibit transport of a reaction product. The thickness of the film can be about 0.0002 to about 0.05 mm, desirably about 0.005 to about 0.04 mm and preferably about 0.01 to about 0.025 mm. FIG. 6 shows CO₂ permeability of a polycarbonate film with thickness at 75° F. and 0% relative humidity, where permeability (P) equals cc/100 in²·atm·day

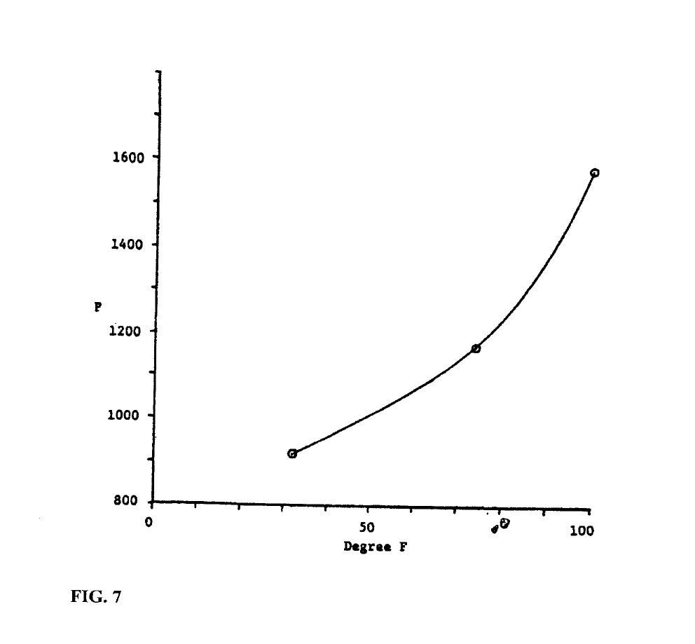

Temperature is another variable that can affect film permeability. FIG. 7 shows the effect of temperature on the permeability of 1 mil blown polycarbonate film at constant relative humidity (RH). FIG. 7 shows permeability versus thickness at 75° F. and 0% relative humidity where P equals cc/100 in²·atm·day. Accordingly, the CHTS method can comprise reacting a reactant at a temperature of about 0 to about 150° C., desirably about 50 to about 140° C. and preferably about 75 to about 125° C.

In one embodiment, the invention is applied to study a process for preparing diaryl carbonates. Diaryl carbonates such as diphenyl carbonate can be prepared by reaction of hydroxyaromatic compounds such as phenol with oxygen and carbon monoxide in the presence of a catalyst composition comprising a Group VIIIB metal such as palladium or a compound thereof, a bromide source such as a quaternary ammonium or hexaalkylguanidinium bromide and a polyaniline in partially oxidized and partially reduced form. The invention can be applied to screen for a catalyst to prepare a diaryl carbonate by carbonylation.

Various methods for the preparation of diaryl carbonates by a carbonylation reaction of hydroxyaromatic compounds with carbon monoxide and oxygen have been disclosed. The carbonylation reaction requires a rather complex catalyst. Reference is made, for example, to Chaudhari et al., U.S. Pat. 5,917,077. The catalyst compositions described therein comprise a Group VIIIB metal (i.e., a metal selected from the group consisting of ruthenium, rhodium, palladium, osmium, iridium and platinum) or a complex thereof.

The catalyst material also includes a bromide source. This may be a quaternary ammonium or quaternary phosphonium bromide or a hexaalkylguanidinium bromide. The guanidinium salts are often preferred; they include the α,ω-bis(pentaalkylguanidinium)alkane salts. Salts in which the alkyl groups contain 2-6 carbon atoms and especially tetra-n-butylammonium bromide and hexaethylguanidinium bromide are particularly preferred.

Other catalytic constituents are necessary in accordance with Chaudhari et al. The constituents include inorganic cocatalysts, typically complexes of cobalt(II) salts with organic compounds capable of forming complexes, especially pentadentate complexes. Illustrative organic compounds of this type are nitrogen-heterocyclic compounds including pyridines, bipyridines, terpyridines, quinolines, isoquinolines and biquinolines; aliphatic polyamines such as ethylenediamine and tetraalkylethylenediamines; crown ethers; aromatic or aliphatic amine ethers such as cryptanes; and Schiff bases. The especially preferred inorganic cocatalyst in many instances is a cobalt(II) complex with bis-3-(salicylalamino)propylmethylamine.

Organic cocatalysts may be present. These cocatalysts include various terpyridine, phenanthroline, quinoline and isoquinoline compounds including 2,2′:6′,2″ -terpyridine, 4-methylthio-2,2′:6′,2″ -terpyridine and 2,2′:6′,2″ -terpyridine N-oxide, 1,10-phenanthroline, 2,4,7,8-tetramethyl-1,1 0-phenanthroline, 4,7-diphenyl-1,10, phenanthroline and 3,4,7,8-tetramethy-1,1 0-phenanthroline. The terpyridines and especially 2,2′:6′,2″ -terpyridine are preferred.

Another catalyst constituent is a polyaniline in partially oxidized and partially reduced form.

Any hydroxyaromatic compound may be employed. Monohydroxyaromatic compounds, such as phenol, the cresols, the xylenols and p-cumylphenol are preferred with phenol being most preferred. The method may be employed with dihydroxyaromatic compounds such as resorcinol, hydroquinone and 2,2-bis(4-hydroxyphenyl)propane or “bisphenol A,” whereupon the products are polyearbonates.

Other reagents in the carbonylation process are oxygen and carbon monoxide, which react with the phenol to form the desired diaryl carbonate.

These and other features will become apparent from the following detailed discussion, which by way of example without limitation describes a preferred embodiment of the present invention.

### EXAMPLE

This example illustrates the identification of an active and selective catalyst for the production of aromatic carbonates. The procedure identifies the best catalyst from within a complex chemical space, where the chemical space is defined as an assemblage of all possible experimental conditions defined by a set of variable parameters such as formulation ingredient identity or amount.

In this Example, a reactor plate is designed to provide a rate of diffusion of reactant gas through a polymer membrane greater than the rate of reaction of the gas to form the desired product. The desired reaction rate of the catalyst is 1 gram-mole/liter-hour. Each cell in the array of the plate is 5 mm in diameter and 1 mm thick, with 0.01 mm film making up the top and bottom of each cell as illustrated in FIG. 4. This design provides a cell volume of 20 mm³ and a film area of 40 mm².

The plate is prepared for reaction by providing a preformed 86×126 mm piece of 1 mm polycarbonate substrate with an 8×12 array of 5-mm holes and heat sealing a piece of 86×126 mm 0.01 mm thick polycarbonate film to the substrate bottom. Twenty (20) microliters of premixed catalyst solution is delivered to each cell. A second 86×126 mm piece of 0.01 mm polycarbonate film is heat sealed to the top of the plate substrate.

The subsequent reaction is run at 100° C. and at a partial pressure of 10 atmospheres of O₂. Permeability of the film to oxygen at 100° C. is calculated to be 5×10⁻⁹ cc(STP)-mm/cm²-sec-cmHg. Oxygen flow through the film is calculated as 2.44×10⁻⁰⁵ gram/moles-hour to provide an oxygen delivery rate to the 20 mm³ (2×10⁻⁵ liters) reaction volume of 1.22 g-mols/liter-hour. Formulation parameters are given in TABLE 1.

TABLE 1

| | Formulation Type Parameter | Formulation Amount |
|---|---|---|
| | Variation | Parameter Variation |
| Precious | Held Constant | Held Constant |
| metal | | |
| catalyst | | |
| Transition | Ti, V, Cr, Mn, Fe, Co, Ni, | 5 (as molar ratios to |
| Metal | Cu (as their acetylacetonates) | precious metal catalyst) |
| Cocatalyst | | |
| (TM) | | |
| Lanthanide | La, Ce, Eu, Gd (as their | 5 (as molar ratios to |
| Metal | acetylacetonates) | precious metal catalyst) |
| Cocatalyst | | |
| (LM) | | |
| Cosolvent | Dimethylformamide (DMFA), | 500 (as molar ratios to |
| (CS) | Dimethylacetamide (DMAA), | precious metal catalyst) |
| | Diethyl acetamide (DEAA) | |
| Hydroxy- | Held constant | Sufficient added to achieve |
| aromatic | | constant sample volume |
| compound | | |

The size of the initial chemical space defined by the parameters of TABLE 1 is 96 possibilities. This is a large experimental space for a conventional technique. However, the experiment can be easily conducted according to the present invention to determine optimal compositions. The space is explored using a full factorial design. A full factorial design of experiment (DOE) measures the response of every possible combination of factors and factor levels. These responses can be analyzed to provide information about every main effect and every interaction effect. The design is given in TABLE 2, below.

In this experiment, each metal acetylacetonate and each cosolvent were made up as stock solutions in phenol. Ten ml of each stock solution are produced by manual weighing and mixing. For each sample, an appropriate quantity of each stock solution is then combined using a Hamilton MicroLab 4000 laboratory robot into a single 2-ml vial. The mixture is stirred using a miniature magnetic stirrer. Then 20 microliter aliquots are measured out by the robot to individual cells in the array. After the aliquots are distributed, the upper film is heat sealed to the substrate.

The assembled reactor plate is then placed in an Autoclave Engineers 1-gallon autoclave, which is then pressurized to 1500 psi (100 atm) with a 10% O₂ in CO mixture. This provides a 10 atm oxygen partial pressure. the autoclave is heated to 100° C. for two hours, cooled, depressurized and the array removed. Raman spectrum of each product is taken by focussing an argon ion laser 38 (Spectra Physics 2058) on a cell and detecting the inelastically scattered light with an Acton Spectra-Pro 3001 spectrophotometer 36.

Performance in this example is expressed numerically as a catalyst turnover number or TON. TON is defined as the number of moles of aromatic carbonate produced per mole of charged palladium catalyst. The performance of each of the runs is given in the column “TON” of TABLE 2.

TABLE 2

| | Transition | Lanthanide | | |
|---|---|---|---|---|
| | Metal (TM) | Metal (LM) | Cosolvent | |
| Run | Cocatalyst | Cocatalyst | (CS) | TON |
| 1 | Mn | Gd | DEAA | 555.1078 |
| 2 | Cu | La | DMAA | 456.5777 |
| 3 | Mn | Ce | DMAA | 513.6325 |
| 4 | Ti | Gd | DEAA | 400.5089 |
| 5 | V | Eu | DMFA | 587.5912 |
| 6 | Mn | La | DMAA | 1750.03 |
| 7 | Ti | Ce | DEAA | 292.4069 |
| 8 | Cr | Eu | DMAA | 625.9431 |
| 9 | V | Ce | DMFA | 665.1948 |
| 10 | Fe | Eu | DMFA | 332.9006 |
| 11 | Ti | Eu | DMFA | 679.5486 |
| 12 | Fe | La | DEAA | 468.5033 |
| 13 | Co | Ce | DEAA | 257.2479 |
| 14 | Cu | Eu | DMAA | 468.7711 |
| 15 | Ni | Ce | DMFA | 433.6684 |
| 16 | Co | Gd | DMAA | 485.2293 |
| 17 | Cu | Gd | DEAA | 342.2256 |
| 18 | Cu | Gd | DMFA | 506.5736 |
| 19 | Mn | Eu | DMFA | 356.3573 |
| 20 | Co | La | DMFA | 545.6339 |
| 21 | Ni | Gd | DMFA | 483.2507 |
| 22 | V | Gd | DMFA | 590.907 |
| 23 | Ti | La | DEAA | 885.7548 |
| 24 | Cr | Eu | DEAA | 344.2193 |
| 25 | Mn | Gd | DMFA | 338.4866 |
| 26 | Fe | Ce | DMFA | 474.0333 |
| 27 | Ni | Eu | DMFA | 758.6696 |
| 28 | Mn | Ce | DMFA | 625.6508 |
| 29 | Cr | Gd | DMFA | 603.5539 |
| 30 | Cr | Eu | DMFA | 249.9745 |
| 31 | Co | Eu | DEAA | 431.0617 |
| 32 | Mn | Gd | DMAA | 372.3904 |
| 33 | Ni | Gd | DMAA | 652.7145 |
| 34 | Cu | Ce | DMAA | 352.7221 |
| 35 | Ni | Eu | DEAA | 459.774 |
| 36 | Co | Gd | DEAA | 472.6578 |
| 37 | Fe | La | DMFA | 472.984 |
| 38 | V | La | DMAA | 858.9171 |
| 39 | V | Eu | DMAA | 416.1047 |
| 40 | Cu | La | DEAA | 345.512 |
| 41 | Cr | La | DMFA | 552.11 |
| 42 | Cu | Eu | DEAA | 250.3933 |
| 43 | Cr | La | DEAA | 417.1977 |
| 44 | Mn | La | DEAA | 1291.111 |
| 45 | V | Gd | DEAA | 490.6305 |
| 46 | Co | Gd | DMFA | 452.9355 |
| 47 | V | Gd | DMAA | 413.9911 |
| 48 | Cu | Gd | DMAA | 683.2233 |
| 49 | Fe | Ce | DEAA | 276.7799 |
| 50 | Co | La | DEAA | 390.3853 |
| 51 | Ti | Gd | DMAA | 390.6338 |
| 52 | Ni | La | DMAA | 673.2558 |
| 53 | Mn | Ce | DEAA | 360.0271 |
| 54 | V | Ce | DMAA | 650.6003 |
| 55 | V | La | DMFA | 848.4497 |
| 56 | Cu | La | DMFA | 476.2182 |
| 57 | Cr | Gd | DMAA | 427.1539 |
| 58 | Co | Ce | DMFA | 468.8664 |
| 59 | V | La | DEAA | 743.0518 |
| 60 | Co | Eu | DMAA | 364.7413 |
| 61 | Fe | Eu | DMAA | 572.7474 |
| 62 | V | Eu | DEAA | 459.1624 |
| 63 | Ti | La | DMFA | 778.1048 |
| 64 | Ni | Gd | DEAA | 522.5839 |
| 65 | Fe | Gd | DMAA | 340.3491 |
| 66 | Ni | La | DMFA | 733.7841 |
| 67 | Cr | La | DMAA | 613.4944 |
| 68 | V | Ce | DEAA | 295.7852 |
| 69 | Ni | Eu | DMAA | 868.0304 |
| 70 | Fe | La | DMAA | 559.6479 |
| 71 | Fe | Gd | DMFA | 592.372 |
| 72 | Cr | Ce | DEAA | 326.6567 |
| 73 | Cr | Ce | DMAA | 417.9809 |
| 74 | Cu | Ce | DEAA | 267.8915 |
| 75 | Ni | Ce | DEAA | 262.121 |
| 76 | Ni | Ce | DMAA | 554.9479 |
| 77 | Cr | Ce | DMFA | 495.3985 |
| 78 | Ni | La | DEAA | 451.5785 |
| 79 | Ti | Eu | DMAA | 877.8409 |
| 80 | Fe | Ce | DMAA | 612.9162 |
| 81 | Mn | Eu | DMAA | 644.8604 |
| 82 | Fe | Gd | DEAA | 521.141 |
| 83 | Fe | Eu | DEAA | 457.5463 |
| 84 | Mn | La | DMFA | 1650.954 |
| 85 | Ti | Eu | DEAA | 450.2065 |
| 86 | Ti | Ce | DMAA | 512.3347 |
| 87 | Cu | Ce | DMFA | 324.8884 |
| 88 | Ti | Gd | DMFA | 747.381 |
| 89 | Co | Ce | DMAA | 242.6424 |
| 90 | Co | La | DMAA | 366.3668 |
| 91 | Co | Eu | DMFA | 474.389 |
| 92 | Ti | Ce | DMFA | 374.0002 |
| 93 | Cu | Eu | DMFA | 549.2309 |
| 94 | Cr | Gd | DEAA | 279.3706 |
| 95 | Ti | La | DMAA | 634.0476 |
| 96 | Mn | Eu | DEAA | 350.5033 |

The results are analyzed using a “General Linear Model” routine in Minitab software. The routine is set to calculate an Analysis of Variance (ANOVA) for all main effects and 2-way interactions. The ANOVA is given in TABLE 3. In TABLE 3, Sources of Variation are potentially significant factors and interactions. Degrees of Freedom are a measure of the amount of information available for each source. Adjusted Sums of Squares are the squares of the deviations caused by each source. Adjusted Mean Squares are Adjusted Sums/Degrees of Freedom. The F Ratio is the Adjusted Mean Square for each Source/Adjusted Mean Square for Error. The F ratio is compared to a standard table to determine its statistical significance at a given probability (0.001 or 0.1% in this case).

TABLE 3

| Source of | Degrees of | Adjusted Sums of | Adjusted Mean | | Significant |
|---|---|---|---|---|---|
| Variation | Freedom | Squares | Squares | F Ratio | at P < 0.001 |
| TM | 7 | 1243723 | 177675 | 9.84 | Yes |
| LM | 3 | 973525 | 324508 | 17.98 | Yes |
| CS | 2 | 896969 | 448484 | 24.84 | Yes |
| TM * LM | 21 | 1754525 | 83549 | 4.63 | Yes |
| TM * CS | 14 | 353434 | 25245 | 1.4 | No |
| LM * CS | 6 | 205012 | 34169 | 1.89 | No |
| Error | 42 | 758191 | 18052 | | |
| Total | 95 | | | | |

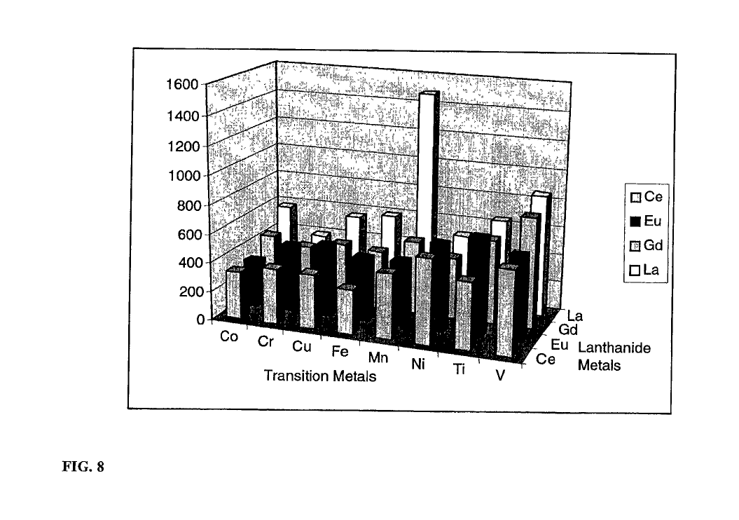

The column “Significant at P<0.001” indicates that a TM*LM (transition metal *lanthanide metal) interaction has a significant effect on TON. These interactions are also illustrated in FIG. 8, which shows that interaction of Mn and La have a strong positive influence on the TON.

While preferred embodiments of the invention have been described, the present invention is capable of variation and modification and therefore should not be limited to the precise details of the Example. The invention includes changes and alterations that fall within the purview of the following claims.

What is claimed is:

1. A reactor plate, comprising:
a substrate with an array of reaction cells; and
a permeable film covering at least one of the cells to selectively permit transport of a reactant gas into the one cell while preventing transport of a reaction product out of the cell.

2. The reactor plate of claim 1, wherein the film is characterized by a diffusion coefficient of about 5×10⁻¹⁰ to about 5×10−7 cc(STP)-mm/cm²-sec-cmHg.

3. The reactor plate of claim 1, wherein the film is characterized by a diffusion coefficient of about 1×10⁻⁹ to about 1×10⁻⁷ cc(STP)-mm/cm²-sec-cmHg.

4. The reactor plate of claim 1, wherein the film is characterized by a diffusion coefficient of about and preferably about 2×10⁻⁸ to about 2×10⁻⁶ cc(STP)-mm/cm²-sec-cmHg.

5. The reactor plate of claim 1, wherein the film is about 0.0002 to about 0.05 mm thick.

6. The reactor plate of claim 1, wherein the film is about 0.005 to about 0.04 mm thick.

7. The reactor plate of claim 1, wherein the film is, desirably about 0.01 to about 0.025 mm thick.

8. The reactor plate of claim 1, wherein the film is a polycarbonate, perfluoroethylene, polyamide, polyester, polypropylene or polyethylene.

9. The reactor plate of claim 1, wherein the film is a polycarbonate, PET or polypropylene.

10. The reactor plate of claim 1, wherein the film is a monofilm, coextrusion, composite or laminate.

11. The reactor plate of claim 1, wherein the film selectively admits transport of a reactant and prohibits transport of a reaction product.

12. The reactor plate of claim 1, wherein the film selectively admits transport of oxygen and carbon monoxide and prohibits transport of a diaryl carbonate.

13. The reactor plate of claim 1, wherein the at least one cell is a shallow cell.

14. The reactor plate of claim 1, wherein the at least one cell is a cell with two opposing walls comprising permeable film.

15. The reactor plate of claim 1, wherein the at least one cell is a cell is formed from a polycarbonate substrate with two opposing walls comprising permeable polycarbonate film

16. The reactor plate of claim 1, wherein the at least one cell is a concave bottomed cell with permeable film cover.

17. A method, comprising:
providing a reactor plate comprising a substrate with an array of reaction cells, at one least one cell of the array comprising a cavity and a permeable film cover; and
conducting a combinatorial high throughput screening (CHTS) method with the reactor plate.

18. The method of claim 17, wherein the CHTS method comprises a step of (a) reacting a reactant under a set of catalysts or reaction conditions; and (b) evaluating a set of products of the reacting step.

19. The method of claim 17, comprising providing a cell according to permeability of the film and robustness and rate of the reacting step.

20. The method of claim 17, comprising providing a cell so that rate of diffusion of gas through the membrane is greater than the rate of gas uptake of the reaction in the reacting step.

21. The method of claim 17, wherein the CHTS method comprises (A) an iteration of steps of (i) selecting a set of reactants; (ii) reacting the set and (iii) evaluating a set of products of the reacting step and (B) repeating the iteration of steps (i), (ii) and (iii) wherein a successive set of reactants selected for a step (i) is chosen as a result of an evaluating step (iii) of a preceding iteration.

22. The method of claim 17, wherein the CHTS method comprises (A) (i) simultaneously reacting reactants, (ii) identifying a multiplicity of tagged products of the reaction and (B) evaluating the identified products after completion of a single or repeated iteration (A).

23. The method of claim 17, wherein the CHTS method comprises (a) reacting a reactant under a set of catalysts or reaction conditions; (b) evaluating a set of products of the reacting step; and reiterating (a) according to results of the evaluating (b).

24. The method of claim 17, wherein the CHTS method comprises (a) reacting a reactant at a temperature of about 0 to about 150° C.

25. The method of claim 17, wherein the CHTS method comprises (a) reacting a reactant at a temperature of about 50 to about 140° C.

26. The method of claim 17, wherein the CHTS method comprises (a) reacting a reactant at a temperature of about 75 to about 125° C.

27. The method of claim 17, wherein the CHTS method comprises effecting parallel chemical reactions of reactants or catalysts within reaction cells of the array.

28. The method of claim 17, wherein the CHTS method comprises effecting parallel chemical reactions on a micro scale on reactants or catalysts within reaction cells of the array.

29, The method of claim 17, wherein the CHTS method comprises effecting parallel chemical reactions on catalyst systems within reaction cells of the array with reactants that permeate through the film cover.

30. The method of claim 29, wherein at least one catalyst system comprises a Group VIIIB metal.

31. The method of claim 29, wherein at least one catalyst system comprises palladium.

32. The method of claim 29, wherein at least one catalyst system comprises a halide composition.

33. The method of claim 29, wherein at least one catalyst system comprises an inorganic co-catalyst.

34. The method of claim 29, wherein at least one catalyst system comprises a combination of inorganic co-catalysts.

35. The method of claim 17, further comprising depositing a reactant within the at least one cell and effecting a chemical reaction of the reactant with carbon monoxide and oxygen that permeates through the film.

36. The method of claim 35, wherein the film is a polycarbonate, PET or polypropylene.
